(19) United States
(12) Patent Application Publication
(10) Pub. No.: US 2020/0360247 A1
(43) Pub. Date: Nov. 19, 2020

(54) WHITE ACTIVATED CHARCOAL TOOTH WHITENING COMPOUND

(71) Applicant: TOTAL CLEAN HOLDINGS LLC, New York, NY (US)

(72) Inventor: Martin Giniger, Delray Beach, FL (US)

(21) Appl. No.: 16/869,588

(22) Filed: May 7, 2020

Related U.S. Application Data
(60) Provisional application No. 62/847,882, filed on May 14, 2019.

(51) Int. Cl. A61K 8/22; A61K 8/34; A61K 8/60; A61K 8/92; A61K 8/9706; A61Q 11/00

(52) U.S. Cl. CPC A61K 8/22; A61K 8/345; A61Q 11/00; A61K 8/922; A61K 8/9706; A61K 8/60

(57) ABSTRACT

The present invention relates to one or more oral care compositions utilizing white activated charcoal, including a spot treatment whitening compound minimally comprising white activated charcoal, a peroxide, and a carbomer. The present invention further relates to a highly effective spot treatment whitening compound further comprising one or more of a sweetener, a thickening agent, a flavoring agent, and a solvent base.

### CROSS REFERENCE TO RELATED APPLICATIONS

The present application claims the benefit of U.S. Provisional Application 62/847,882 filed May 14, 2019.

### FIELD OF THE INVENTION

The field of the present invention generally relates to tooth whitening products.

### BACKGROUND OF THE INVENTION

Some might find the choice of toothpaste and other oral care products to be an unremarkable decision made at one or both sleepy times of our busy day, but in fact there have been great advances in this field of technology time and again. For two well-known examples, consider the introduction of fluoride salts and of artificial sweeteners—the first introduced an active technology to fight cavity formation and growth by artificially strengthening enamel on teeth and the second drastically improved the frequency and consistency of brushing—merely by improving the brushing experience without the use of natural sugars (which are known to feed cavity-forming bacteria and fuel plaque formation).

Black activated charcoal has only recently become a popular ingredient in oral care products. Even as more products using black activated charcoal have been introduced into the market, it has been found that black activated charcoal is very messy and inconvenient. It blackens the brusher's mouth and can leave unsightly stains on toothbrushes, bathroom sinks, mirrors and floors.

Novel inventions can be highly effective and yet still struggle to break through into widespread use. Sometimes an invention relies on a new and unfamiliar technology that raises concerns about safety (such as the introduction of air travel) or has unproven efficacy (such as the introduction of new surgical implant device). Activated charcoal is a very old technology, providing a unique adsorbant profile in the micro pitting—known to attract and retain (i.e. adsorb) a variety of contaminants—ranging from staining materials to volatile compounds that cause bad breath. Active charcoal has been successfully added to toothpaste as a whitening agent. Despite the impressive research demonstrating an efficacy which rivals that of chemical bleaching agents, harsh and often toxic chemical bleaching agents remain the typical consumer's preferred choice of whitener.

The present invention utilizes an optically white activated charcoal which behaves no differently from the more familiar black form. Because there is widespread suspicion of chemical additives and a growing demand for safe and natural alternatives, the present invention takes a novel approach and has the potential to transform dental care in a manner similar to that of fluoride by breaking through reluctant consumer uptake of a highly effective product, which has previously been dirty and messy to use.

The present invention introduces the application of optically white activated charcoal to replace the familiar activated charcoal (with dark appearance) known in prior art—to whiten teeth naturally without the mess. One embodiment of the present invention sets forth a spot treatment whitening compound for use in oral care. In particular, the spot treatment can be loaded into an applicator (or a “Whitening Pen”). In a further embodiment still because whitening can have side effects of sensitivity of teeth with continuous use as well as potential irritation of gums, a gum soothing ingredient (such as sodium stannate) is further compounded in. Another further embodiment still provides for a comprehensive whitening treatment methodology combining regular brushing using said complete toothpaste solution combined with daily ad hoc treatment with said Whitening Pen (preferably daily). In a further embodiment still, the Whitening Pen is equipped with a hollow silicone brush tip to allow a user to both apply and rub the spot treatment whitening compound into their teeth.

### SUMMARY OF THE INVENTION

In one preferred embodiment of the invention, a spot treatment whitening compound minimally including white activated charcoal is compounded with a peroxide and a carbomer.

In a further preferred embodiment of the invention, a spot treatment whitening compound minimally including white activated charcoal is compounded with peroxide and a carbomer in a solvent base.

In a further preferred embodiment of the invention, the solvent base is selected from the group consisting of glycerin and water.

In a further preferred embodiment of the invention, the solvent base is Glycerin.

In a further preferred embodiment of the invention, the spot treatment whitening compound minimally including white activated charcoal is compounded with between 1% w/w and 70% w/w of Peroxide and also a carbomer.

In a further preferred embodiment of the invention, the peroxide above is selected from the group consisting of hydrogen peroxide, sodium perborate, carbamide peroxide, alkali metal peroxide, alkali metal percarbonate, and alkali metal perborate.

In a further preferred embodiment of the invention the peroxide is hydrogen peroxide.

In a further preferred embodiment of the invention the spot treatment whitening compound is compounded with the following ingredients as set forth in Table 1: Glycerin, Hydrogen Peroxide (in solution with water), White Activated Charcoal, Xylitol, Irish Moss, Peppermint Oil, Sodium Stannate.

### Table 1

| Ingredient | Function |
|---|---|
| Glycerin, USP | SOLVENT |
| Hydrogen Peroxide | TOOTH WHITENER |
| (in solution with water) | |
| White Activated Charcoal | STAIN REMOVER & WHITENER |
| Xylitol, USP | SWEETENER |
| Irish Moss | THICKENER |
| Peppermint Oil, USP | FLAVOR |
| Sodium Stannate, USP | SOOTHES GUMS & STABILIZER |

In another further embodiment, a method for comprehensive whitening treatment combining regular (at least twice daily) brushing with said complete toothpaste solution combined with intermittent (ad hoc, preferably once daily) whitening treatment applying spot treatment whitening compound using a Whitening Pen.

In a further embodiment still, the Whitening Pen is an applicator pen that is used once daily after brushing.

In a further embodiment still, a comprehensive tooth whitening method is disclosed as follows: brush teeth (for two minutes using Whitening Toothpaste, expectorate and rinse with water, ideally twice per day), wipe teeth dry (using a hygienic cloth or paper tissue), dispense spot treatment whitening compound (rotate base of pen (White Birch Activated White Charcoal) to dispense whitening compound through brush tip), apply whitening compound (hold a wide smile and apply the whitening compound to the front surface of all teeth visible in users smile), scrub in compound (by brushing onto front teeth with the silicon brush for one minute, focusing on areas with visible staining), rinse (using water), refrain (from eating, drinking or smoking) for 30 minutes following treatment.

The embodiments of the invention in which an exclusive property or privilege is claimed are defined as follows:

1. An oral care composition comprising a white activated charcoal, a peroxide, and a carbomer.

2. The oral care composition according to claim 1, further comprising a solvent base.

3. The oral care composition according to claim 2, wherein the solvent base is water.

4. The oral care composition according to claim 2, wherein the solvent base is glycerin.

5. The oral care composition according to claim 1, wherein the peroxide is hydrogen peroxide (in solution with water).

6. The oral care composition according to claim 1, wherein the carbomer is sodium stannate.

7. The oral care composition according to claim 1, further comprising xylitol.

8. The oral care composition according to claim 1, further comprising irish moss.

9. The oral care composition according to claim 1, further comprising peppermint oil.

10. The oral care composition according to claim 5, wherein the carbomer is sodium stannate and further comprising xylitol, irish moss, peppermint oil and glycerin.

11. A spot treatment whitening compound comprising: (a) from 1.0% to 70.0% by weight of a peroxide; (b) a carbomer; (c) a white activated charcoal, wherein the percentage by weight is relative to the total weight of the composition, and wherein the composition is for whitening teeth.

12. The spot treatment whitening compound according to claim 11, further comprising a solvent base.

13. The spot treatment whitening compound according to claim 12, wherein the solvent base is glycerin.

14. The spot treatment whitening compound according to claim 11, wherein the peroxide is selected from the group consisting of hydrogen peroxide, sodium perborate, carbamide peroxide, alkali metal peroxide, alkali metal percarbonate, and alkali metal perborate.

15. The spot treatment whitening compound according to claim 11, wherein the peroxide is hydrogen peroxide.

16. The spot treatment whitening compound according to claim 11, wherein the carbomer is sodium stannate.

17. The spot treatment whitening compound according to claim 11, further comprising xylitol.

18. The spot treatment whitening compound according to claim 11, further comprising irish moss.

19. The spot treatment whitening compound according to claim 11, further comprising peppermint oil.

20. The spot treatment whitening compound according to claim 13, wherein the peroxide is hydrogen peroxide (in solution with water), wherein the carbomer is sodium stannate, and further comprising xylitol, irish moss, and peppermint oil.
